(19) United States
(12) Patent Application Publication
(10) Pub. No.: US 2026/0335358 A1
(43) Pub. Date: Oct. 29, 2026

(54) PRUSSIAN BLUE COMPOSITE AND PREPARATION METHOD AND USE THEREOF

(71) Applicant: The Second Hospital of Dalian Medical University, Dalian City (CN)

(72) Inventors: Shuai Wang, Dalian (CN); Jianbin Zhang, Dalian City (CN); Jing Li, Dalian City (CN); Xi Wan, Dalian City (CN); Yanbing Song, Dalian City (CN); Jianing Li, Dalian City (CN)

(21) Appl. No.: 19/651,097

(22) Filed: Apr. 17, 2026

(30) Foreign Application Priority Data
Apr. 27, 2025 (CN) 202510537697.4

(51) Int. Cl. A61K 33/26; A61K 9/1277; A61K 9/51; A61K 33/22; A61P 27/02

(52) U.S. Cl. CPC A61K 33/26; A61K 9/1277; A61K 9/5138; A61K 9/5192; A61K 33/22; A61P 27/02

(57) ABSTRACT

Provided are a Prussian blue composite, and a preparation method and use thereof. The Prussian blue composite includes hollow mesoporous Prussian blue nanoparticles, a liposome coating the hollow mesoporous Prussian blue nanoparticles, and an ammonia borane complex loaded on the hollow mesoporous Prussian blue nanoparticles.

### CROSS REFERENCE TO RELATED APPLICATION

This patent application claims the benefit and priority of Chinese Patent Application No. 202510537697.4 filed with the China National Intellectual Property Administration on Apr. 27, 2025, the disclosure of which is incorporated by reference herein in its entirety as part of the present application.

### TECHNICAL FIELD

The present disclosure belongs to the technical field of biomedicines, in particular relating to a Prussian blue composite, and a preparation method and use thereof.

### BACKGROUND

Dry eye syndrome is a complex ocular surface disease with a high prevalence, associated with corneal damage, excessive oxidative stress, and inflammation. Current treatment strategies involve hydrogen therapy, primarily employing an ammonia borane complex (NH₃BH₃, abbreviated as AB). The ammonia borane complex is a molecule having low toxicity, strong acid reactivity, and high hydrogen storage capacity, and can release hydrogen very slowly in pH = 7 ± 0.1 environments at room temperature and rapidly in acidic environments, resulting in the presence of a large amount of H₂ within cells, which effectively blocks the mitochondrial respiratory chain, reduces adenosine triphosphate (ATP) production, and further inhibits intracellular reactive oxygen species (ROS) expression, thereby alleviating dry eye syndrome, but the alleviation effect is limited. Therefore, how to improve the ammonia borane complex to enhance the effect in relieving dry eye syndrome has become an urgent technical problem to be solved in the art.

### SUMMARY

The present disclosure is to provide a Prussian blue composite, and a preparation method and use thereof. In the present disclosure, the Prussian blue composite can slowly release hydrogen to the inflammatory sites of dry eye syndrome, while alleviating damages caused by oxidative stress, thereby effectively relieving the symptoms of dry eye syndrome.

To achieve the foregoing object, the present disclosure provides the following technical solutions.

The present disclosure provides a Prussian blue composite, comprising hollow mesoporous Prussian blue nanoparticles, a liposome coating the hollow mesoporous Prussian blue nanoparticles, and an ammonia borane complex loaded on the hollow mesoporous Prussian blue nanoparticles.

In some embodiments, the hollow mesoporous Prussian blue nanoparticles are prepared by a process comprising:
mixing Prussian blue nanoparticles, a first stabilizer, and a first acidic solution to obtain a mixed solution, and subjecting the mixed solution to etching to obtain the hollow mesoporous Prussian blue nanoparticles.

In some embodiments, the etching is conducted at a temperature of 130°C to 150°C for 3 hours to 8 hours.

The present disclosure further provides a method for preparing the Prussian blue composite as described in the above technical solutions, comprising the steps of:
(1) mixing a liposome raw material and an organic solvent to obtain a lipid solution;
(2) subjecting the lipid solution obtained in step (1) to rotary evaporation to obtain a lipid film;
(3) mixing the lipid film obtained in step (2), the hollow mesoporous Prussian blue nanoparticles, and water, and conducting coating to obtain liposome-coated hollow mesoporous Prussian blue nanoparticles; and
(4) mixing the liposome-coated hollow mesoporous Prussian blue nanoparticles obtained in step (3), a buffer solution, and an ammonia borane complex, and conducting loading to obtain the Prussian blue composite.

In some embodiments, the liposome raw material in step (1) comprises lecithin, cholesterol and DSPE-mPEG2000 (1,2-distearoyl-sn-glycero-3-phosphoethanolamine-N-[methoxy(polyethylene glycol)-2000]).

In some embodiments, in step (3), a mass ratio of the lipid film to the hollow mesoporous Prussian blue nanoparticles is in a range of (0.5–4) : 1.

In some embodiments, in step (3), the coating is conducted at a temperature of 10°C to 30°C for 10 minutes to 30 minutes.

In some embodiments, in step (4), a mass ratio of the ammonia borane complex to the liposome-coated hollow mesoporous Prussian blue nanoparticles is in a range of 1 : (0.2–17).

In some embodiments, in step (4), the loading is conducted at a temperature of 4°C to 25 °C for 24 hours to 72 hours.

The present disclosure further provides use of the Prussian blue composite as described in the above technical solutions or the Prussian blue composite prepared by the method as described in the above technical solutions in preparation of a pharmaceutical composition for relieving the symptoms of dry eye syndrome.

The present disclosure provides a Prussian blue composite, comprising hollow mesoporous Prussian blue nanoparticles, a liposome coating the hollow mesoporous Prussian blue nanoparticles, and an ammonia borane complex loaded on the hollow mesoporous Prussian blue nanoparticles. In the present disclosure, the hollow mesoporous Prussian blue nanoparticles can mimic various enzyme-like activities to scavenge ROS, such as superoxide dismutase (SOD), catalase (CAT), and peroxidase (POD)-like activities, to alleviate inflammatory damage caused by oxidative stress. By loading the ammonia borane complex onto the hollow mesoporous Prussian blue nanoparticles, the ammonia borane complex can sustainably supply hydrogen to the inflammatory sites of dry eye syndrome, thereby alleviating the symptoms of dry eye syndrome in combination with the hollow mesoporous Prussian blue nanoparticles. The liposome can specifically deliver the Prussian blue composite to specific cells or tissues, thereby improving the targeting and efficacy of drugs, and further alleviating the symptoms of dry eye syndrome. Experimental results show that in the present disclosure, the Prussian blue composite can effectively improve the symptoms and corneal morphology in mice with dry eye syndrome, and can also reduce the ROS expression level of HCECs in dry eyes, thus effectively improving the symptoms of dry eye syndrome.

### BRIEF DESCRIPTION OF THE DRAWINGS

FIG. 1 shows a transmission electron microscope image of the HPB in Example 1;

FIG. 2 shows a transmission electron microscope image of the HPB@Lip in Example 1;

FIG. 3 shows a transmission electron microscope image of the HPB@Lip@AB prepared in Example 1;

FIG. 4 shows an anterior segment photograph of the corneal sodium fluorescein staining in a normal mouse according to an embodiment of the present disclosure;

FIG. 5 shows an anterior segment photograph of the corneal sodium fluorescein staining in a mouse with dry eye syndrome according to an embodiment of the present disclosure;

FIG. 6 shows an anterior segment photograph of the corneal sodium fluorescein staining in a mouse with dry eye syndrome improved by the HPB@Lip in Example 1;

FIG. 7 shows an anterior segment photograph of the corneal sodium fluorescein staining in a mouse with dry eye syndrome improved by the AB in Example 1;

FIG. 8 shows an anterior segment photograph of the corneal sodium fluorescein staining in a mouse with dry eye syndrome improved by the HPB@Lip@AB prepared in Example 1;

FIG. 9 shows the scorings for the corneal sodium fluorescein staining in normal mice and mice with dry eye syndrome, after being applied with normal saline, the HPB@Lip in Example 1, the AB in Example 1, and the HPB@LiP@AB prepared in Example 1, respectively;

FIG. 10 shows the secretion amounts of tears in normal mice and mice with dry eye syndrome, after being applied with normal saline, the HPB@Lip in Example 1, the AB in Example 1, and the HPB@LiP@AB prepared in Example 1, respectively;

FIG. 11 shows the tear break-up time in normal mice and mice with dry eye syndrome, after being applied with normal saline, the HPB@Lip in Example 1, the AB in Example 1, and the HPB@LiP@AB prepared in Example 1, respectively;

FIG. 12 shows an H&E staining image of the cornea in a normal mouse according to an embodiment of the present disclosure;

FIG. 13 shows an H&E staining image of the cornea in a mouse with dry eye syndrome according to an embodiment of the present disclosure;

FIG. 14 shows an H&E staining image of the mouse cornea applied with the HPB@Lip in Example 1;

FIG. 15 shows an H&E staining image of the mouse cornea applied with the AB in Example 1;

FIG. 16 shows an H&E staining image of the mouse cornea applied with the HPB@Lip@AB prepared in Example 1;

FIG. 17 shows a DHE staining image of normal in vitro HCECs according to an embodiment of the present disclosure;

FIG. 18 shows a DHE staining image of in vitro HCECs with dry eye syndrome according to an embodiment of the present disclosure;

FIG. 19 shows a DHE staining image of in vitro HCECs intervened with the HPB@Lip in Example 1;

FIG. 20 shows a DHE staining image of in vitro HCECs intervened with the AB in Example 1;

FIG. 21 shows a DHE staining image of in vitro HCECs intervened with the HPB@Lip@AB prepared in Example 1; and

FIG. 22 shows the quantitative analysis of DHE (dihydroethidium) staining for normal in vitro HCECs, in vitro HCECs with dry eye syndrome, in vitro HCECs intervened with the HPB@Lip in Example 1, in vitro HCECs intervened with the AB in Example 1, and in vitro HCECs intervened with the HPB@Lip@AB prepared in Example 1.

### DETAILED DESCRIPTION OF THE EMBODIMENTS

The present disclosure provides a Prussian blue composite, comprising hollow mesoporous Prussian blue nanoparticles, a liposome coating the hollow mesoporous Prussian blue nanoparticles, and an ammonia borane complex loaded on the hollow mesoporous Prussian blue nanoparticles.

In the present disclosure, there are no special limitations on the source of each raw material, and any commercially available products well known to those skilled in the art can be used.

In the present disclosure, the Prussian blue composite comprises hollow mesoporous Prussian blue nanoparticles. In the present disclosure, the hollow mesoporous Prussian blue nanoparticles can mimic various enzyme-like activities to scavenge ROS, such as superoxide dismutase (SOD), catalase (CAT), and peroxidase (POD)-like activities, to alleviate inflammatory damage caused by oxidative stress, thereby relieving the symptoms of dry eye syndrome in combination with the ammonia borane complex.

In some embodiments of the present disclosure, the hollow mesoporous Prussian blue nanoparticles are prepared by a process comprising:
mixing Prussian blue nanoparticles, a first stabilizer, and a first acidic solution to obtain a mixed solution, and etching the mixed solution to obtain the hollow mesoporous Prussian blue nanoparticles.

In some embodiments of the present disclosure, the Prussian blue nanoparticles each have a particle size of 50 nm to 300 nm.

In some embodiments of the present disclosure, the Prussian blue nanoparticles are prepared by a process comprising: mixing potassium ferricyanide, a second stabilizer, and a second acidic solution to obtain a mixed solution, and subjecting the mixed solution to a hydrothermal reaction to obtain the Prussian blue nanoparticles.

In some embodiments of the present disclosure, the second stabilizer is polyvinylpyrrolidone; the polyvinylpyrrolidone is at least one selected from the group consisting of K15 (corresponding to a viscosity-average molecular weight of 8,000 to 12,000), K30 (corresponding to a viscosity-average molecular weight of 45,000 to 58,000), K60 (corresponding to a viscosity-average molecular weight of 270,000 to 400,000), and K90 (corresponding to a viscosity-average molecular weight of 1,000,000 to 1,500,000). In the present disclosure, the polyvinylpyrrolidone can be adsorbed onto the surface of the Prussian blue nanoparticles, preventing aggregation and agglomeration, thereby maintaining the dispersibility of the Prussian blue nanoparticles.

In some embodiments of the present disclosure, the second acidic solution is hydrochloric acid; the hydrochloric acid has a concentration of 0.005 M to 0.02 M, more preferably 0.01 M to 0.015 M. In the present disclosure, the second acidic solution is used to provide an acidic environment.

In some embodiments of the present disclosure, the potassium ferricyanide in the mixed solution has a concentration of 1 mg/mL to 10 mg/mL; the second stabilizer in the mixed solution has a concentration of 50 mg/mL to 200 mg/mL. In one embodiment, the potassium ferricyanide in the mixed solution has a concentration of 2 mg/mL, 3 mg/mL, 3.3 mg/mL, 4 mg/mL, 5 mg/mL, 6 mg/mL, 7 mg/mL, 8 mg/mL, or 9 mg/mL; the second stabilizer in the mixed solution has a concentration of 75 mg/mL, 100 mg/mL, 150 mg/mL, or 180 mg/mL.

In the present disclosure, there are no special limitations on the operation of mixing the potassium ferricyanide, the second stabilizer, and the second acidic solution; any technical solution for preparing a mixture known to those skilled in the art can be used.

In some embodiments of the present disclosure, the hydrothermal reaction is conducted at a temperature of 75°C to 85°C, more preferably 80°C; the hydrothermal reaction is conducted for 16 hours to 24 hours, more preferably 20 hours. In the present disclosure, by limiting the temperature and time of the hydrothermal reaction within the above ranges, the degree of hydrothermal reaction can be increased, thereby increasing the yield of the Prussian blue nanoparticles.

In the present disclosure, the process further comprises, after the hydrothermal reaction, subjecting a product obtained from the hydrothermal reaction to centrifugation, separation, washing, and drying in sequence to obtain the Prussian blue nanoparticles.

In the present disclosure, there are no special limitations on the operation of the centrifugation; any operation known to those skilled in the art can be used.

In the present disclosure, there are no special limitations on the operation of the separation, and any operation well known to those skilled in the art can be used to obtain a solid.

In the present disclosure, there are no special limitations on the operation of the washing, as long as a pH can be at 7 ± 0.1 after the washing.

In the present disclosure, there are no special limitations on the operation of the drying, as long as a constant weight can be obtained upon the drying.

In some embodiments of the present disclosure, a type of the first stabilizer is the same as that of the second stabilizer, and will not be repeated herein.

In some embodiments of the present disclosure, the first acidic substance is hydrochloric acid; the hydrochloric acid has a concentration of 0.8 M to 1.2 M, more preferably 0.9 M to 1.0 M. In the present disclosure, the first acidic substance is used for etching the Prussian blue nanoparticles to obtain the hollow mesoporous Prussian blue nanoparticles.

In some embodiments of the present disclosure, the Prussian blue nanoparticles in the mixed solution have a concentration of 0.5 mg/mL to 2 mg/mL; the first stabilizer in the mixed solution has a concentration of 3 mg/mL to 10 mg/mL. In one embodiment, the Prussian blue nanoparticles in the mixed solution have a concentration of 0.6 mg/mL, 0.8 mg/mL, 1 mg/mL, 1.2 mg/mL, 1.4 mg/mL, 1.6 mg/mL, or 1.8 mg/mL; and the first stabilizer in the mixed solution has a concentration of 4 mg/mL, 5 mg/mL, 6 mg/mL, 7 mg/mL, 8 mg/mL, or 9 mg/mL.

In the present disclosure, there are no special limitations on the operation of mixing the Prussian blue nanoparticles, the first stabilizer, and the first acidic solution; any technical solution for preparing the mixture known to those skilled in the art can be used.

In some embodiments of the present disclosure, the etching is conducted at a temperature of 130°C to 150°C, more preferably 140°C; and the etching is conducted for 3 hours to 8 hours, more preferably 4 hours to 6 hours. In the present disclosure, by limiting the temperature and time of the etching within the above ranges, the etching effect can be further improved.

In the present disclosure, the process further includes, after the etching, subjecting a product obtained from the etching to centrifugation, separation, washing, and drying in sequence to obtain the hollow mesoporous Prussian blue nanoparticles.

In the present disclosure, there are no special limitations on the operation of the centrifugation; any operation known to those skilled in the art can be used.

In the present disclosure, there are no special limitations on the operation of the separation, and any operation well known to those skilled in the art can be used to obtain a solid.

In the present disclosure, there are no special limitations on the operation of the washing, as long as a pH can be 7 ± 0.1 after the washing.

In the present disclosure, there are no special limitations on the operation of the drying, as long as a constant weight can be obtained upon the drying.

In the present disclosure, the hollow mesoporous Prussian blue nanoparticles exhibit good biocompatibility and possess prominent antioxidant and anti-inflammatory properties.

In the present disclosure, the Prussian blue composite comprises a liposome coating the hollow mesoporous Prussian blue nanoparticles. In the present disclosure, the liposome can specifically deliver the Prussian blue composite to specific cells or tissues, thereby improving the targeting and efficacy of drugs, and further alleviating the symptoms of dry eye syndrome.

In the present disclosure, the Prussian blue composite comprises an ammonia borane complex loaded on the hollow mesoporous Prussian blue nanoparticles. In the present disclosure, the ammonia borane complex is loaded onto the hollow mesoporous Prussian blue nanoparticles, so that the ammonia borane complex can sustainably supply hydrogen to the inflammatory sites of dry eye syndrome, thereby alleviating the symptoms of dry eye syndrome in combination with the hollow mesoporous Prussian blue nanoparticles.

In the present disclosure, the hollow mesoporous Prussian blue nanoparticles can mimic various enzyme-like activities to scavenge ROS, such as superoxide dismutase (SOD), catalase (CAT), and peroxidase (POD)-like activities, to alleviate inflammatory damage caused by oxidative stress. By loading the ammonia borane complex onto the hollow mesoporous Prussian blue nanoparticles, the ammonia borane complex can sustainably supply hydrogen to the inflammatory sites of dry eye, thereby alleviating the symptoms of dry eye syndrome in combination with the hollow mesoporous Prussian blue nanoparticles. The liposome can specifically deliver the Prussian blue composite to specific cells or tissues, thereby improving the targeting and efficacy of drugs, and further alleviating the symptoms of dry eye syndrome.

The present disclosure further provides a method for preparing the Prussian blue composite as described in the above technical solutions, comprising the steps of:
(1) mixing a liposome raw material and an organic solvent to obtain a lipid solution;
(2) subjecting the lipid solution obtained in step (1) to rotary evaporation to obtain a lipid film;
(3) mixing the lipid film obtained in step (2), hollow mesoporous Prussian blue nanoparticles, and water, and conducting coating to obtain liposome-coated hollow mesoporous Prussian blue nanoparticles; and
(4) mixing the liposome-coated hollow mesoporous Prussian blue nanoparticles obtained in step (3), a buffer solution, and an ammonia borane complex, and conducting loading to obtain the Prussian blue composite.

In the present disclosure, a liposome raw material and an organic solvent are mixed to obtain a lipid solution.

In some embodiments of the present disclosure, the liposome raw material comprises lecithin, cholesterol and DSPE-mPEG2000.

In some embodiments of the present disclosure, the lecithin comprises 50% to 70%, more preferably 60%, of a total mass of the lecithin, the cholesterol, and the DSPE-mPEG2000; the cholesterol comprises 10% to 30%, more preferably 20%, of the total mass of the lecithin, the cholesterol, and the DSPE-mPEG2000; the DSPE-mPEG2000 comprises 10% to 30%, more preferably 20%, of the total mass of the lecithin, the cholesterol, and the DSPE-mPEG2000. In the present disclosure, by limiting the contents of the lecithin, the cholesterol, and the DSPE-mPEG2000 to the above ranges, the effect of relieving the symptoms of dry eye syndrome can be further improved; where the lecithin is the main structural component of the liposome; the cholesterol can enhance the stability and rigidity of the liposome; and the DSPE-mPEG2000 can modify the surface of the liposome, increasing the circulation time and stability in vivo.

In some embodiments of the present disclosure, the organic solvent is dichloromethane. In the present disclosure, there are no special limitations on the amount of the organic solvent, as long as each raw material can be completely dissolved.

In the present disclosure, there are no special limitations on the operation of mixing the liposome raw material and the organic solvent, and any technical solution for preparing a mixture known to those skilled in the art can be used.

After obtaining the lipid solution, the present disclosure subjects the lipid solution to rotary evaporation to obtain a lipid film.

The present disclosure does not impose any special limitations on the operation of the rotary evaporation, as long as the organic solvent can be completely evaporated.

In the present disclosure, after obtaining the lipid film, the lipid film, the hollow mesoporous Prussian blue nanoparticles, and water are mixed, and coating is conducted to obtain liposome-coated hollow mesoporous Prussian blue nanoparticles.

In some embodiments of the present disclosure, a mass ratio of the lipid film to the hollow mesoporous Prussian blue nanoparticles is in a range of (0.5–4) : 1. In an embodiment, the mass ratio of the lipid film to the hollow mesoporous Prussian blue nanoparticles is in a range of (1–2) : 1.

In the present disclosure, there are no special limitations on the amount of the inorganic solvent, as long as the raw materials can be completely dissolved.

In the present disclosure, there are no special limitations on the operation of mixing the lipid film, the hollow mesoporous Prussian blue nanoparticles, and water, and any technical solution for preparing a mixture known to those skilled in the art can be used.

In some embodiments of the present disclosure, the coating is conducted at a temperature of 10°C to 30 °C; the coating is conducted for 10 minutes to 30 minutes; and the coating is conducted under ultrasonic conditions. In the present disclosure, by limiting the process parameters of coating within the above-mentioned ranges, the coating effect of liposome can be improved.

In the present disclosure, there are no special limitations on the power of the ultrasound; any power known to those skilled in the art can be used.

In an embodiment, the coating is conducted at a temperature of 15°C, 20°C, or 25 °C; and the coating is conducted for 15 minutes, 20 minutes or 25 minutes.

In the present disclosure, the method further comprises, after the coating, subjecting a product obtained from the coating to centrifugation, separation, and drying in sequence to obtain liposome-coated hollow mesoporous Prussian blue nanoparticles.

In the present disclosure, there are no special limitations on the operation of the centrifugation; any operation known to those skilled in the art can be used.

In the present disclosure, there are no special limitations on the operation of the separation, and any operation well known to those skilled in the art can be used to obtain a solid.

In the present disclosure, there are no special limitations on the operation of the drying, and any operation known to those skilled in the art can be used to dry to constant weight.

In the present disclosure, after the liposome-coated hollow mesoporous Prussian blue nanoparticles are obtained, the liposome-coated hollow mesoporous Prussian blue nanoparticles, a buffer solution, and an ammonia borane complex are mixed, and loading is conducted to obtain the Prussian blue composite.

In some embodiments of the present disclosure, the buffer solution is a PBS (phosphate buffered saline) solution. In the present disclosure, there are no special limitations on the concentration of the buffer solution, and any buffer solution known to those skilled in the art can be used.

In the present disclosure, there are no special limitations on the amount of the buffer solution, as long as the raw materials can be completely dissolved.

In some embodiments of the present disclosure, a mass ratio of the ammonia borane complex to the liposome-coated hollow mesoporous Prussian blue nanoparticles is in a range of 1 : (0.2–17), more preferably 1 : (0.5–15). In an embodiment, the mass ratio of the ammonia borane complex to the liposome-coated hollow mesoporous Prussian blue nanoparticles is 1:0.5, 1:1, 1:2, 1:3, 1:4, 1:5, 1:6, 1:7, 1:8, 1:9, 1:10, 1:11, 1:12, 1:13, or 1:14.

In the present disclosure, there are no special limitations on the operation of mixing the liposome-coated hollow mesoporous Prussian blue nanoparticles, the buffer solution, and the ammonia borane complex, and any technical solution for preparing a mixture known to those skilled in the art can be used.

In some embodiments of the present disclosure, the loading is conducted at a temperature of 4°C to 25°C; and the loading is conducted for 24 hours to 72 hours; the loading is conducted under stirring conditions. In the present disclosure, by limiting the temperature and time of loading within the above ranges, the loading effect can be further improved.

In the present disclosure, there are no special limitations on the stirring rate; any operation known to those skilled in the art can be used.

In the present disclosure, the method further comprises, after the loading, subjecting a product obtained by the loading to centrifugation, separation and drying in sequence to obtain the Prussian blue composite.

In the present disclosure, there are no special limitations on the operation of the centrifugation; any operation known to those skilled in the art can be used.

In the present disclosure, there are no special limitations on the operation of the separation, and any operation well known to those skilled in the art can be used to obtain a solid.

In the present disclosure, there are no special limitations on the operation of the drying, and any operation known to those skilled in the art can be used to dry to a constant weight.

In the present disclosure, the method is simple in process.

The present disclosure further provides use of the Prussian blue composite as described in the above technical solutions or the Prussian blue composite prepared by the method as described in the above technical solutions in preparation of a pharmaceutical composition for relieving the symptoms of dry eye syndrome.

In the present disclosure, there are no special limitations on the operation of using the Prussian blue composite in the preparation of the pharmaceutical composition for relieving the symptoms of dry eye syndrome, and any use operations known to those skilled in the art can be used.

The technical solutions of the present disclosure will be clearly and completely described below with reference to the following examples. Apparently, the described examples are merely a part of, rather than all of, the examples of the present disclosure. All other examples obtained by those of ordinary skill in the art based on the examples of the present disclosure without inventive efforts shall fall within the scope of the present disclosure.

### Example 1

A Prussian blue composite consisted of hollow mesoporous Prussian blue nanoparticles, a liposome coating the hollow mesoporous Prussian blue nanoparticles, and an ammonia borane complex loaded on the hollow mesoporous Prussian blue nanoparticles.

The Prussian blue composite was prepared by a method as follows.
(1) 660 mg of potassium ferricyanide and 15 g of polyvinylpyrrolidone K30 were added to 200 mL of 0.01 M hydrochloric acid with magnetic stirring for 0.5 hours to obtain a mixed solution. The mixed solution was transferred to a high-pressure reactor and subjected to a hydrothermal reaction at 80°C for 20 hours. A resulting product was centrifuged at 12,000 rpm for 5 minutes, separated, washed by using deionized water until pH = 7, and dried to obtain Prussian blue nanoparticles; where a concentration of the potassium ferricyanide in the mixed solution was 3.3 mg/mL; and a concentration of the polyvinylpyrrolidone K30 in the mixed solution was 75 mg/mL.
(2) 200 mg of the Prussian blue nanoparticles obtained in step (1) and 1.0 g of polyvinylpyrrolidone K30 were added to 200 mL of 1.0 M hydrochloric acid with magnetic stirring for 0.5 hours to obtain a mixed solution. The mixed solution was transferred to a high-pressure reactor and subjected to etching at 140°C for 4 hours. A resulting product was centrifuged at 12,000 rpm for 5 minutes, separated, washed by using deionized water until pH = 7, and dried to obtain hollow mesoporous Prussian blue nanoparticles (HPB); where the Prussian blue nanoparticles in the mixed solution were at a concentration of 1 mg/mL; and the first stabilizer in the mixed solution was at a concentration of 5 mg/mL.
(3) 0.6 g of lecithin, 0.2 g of cholesterol, and 0.2 g of DSPE-mPEG2000 were weighed; 10 mL of dichloromethane was added thereto for dissolution to obtain a lipid solution.
(4) The lipid solution obtained in step (3) was added to a round-bottom flask, and dichloromethane was removed by rotary evaporation at 40 °C to obtain a lipid film.
(5) 0.5 g of the hollow mesoporous Prussian blue nanoparticles obtained in step (2) and 50 mL of deionized water were added to the round-bottom flask in step (4), with sonication at 30°C for 25 minutes, and coating was conducted. A resulting mixture was centrifuged at 12,000 rpm for 20 minutes, a resulting supernatant was removed, and a resulting precipitate was freeze-dried under vacuum to obtain liposome-coated hollow mesoporous Prussian blue nanoparticles (HPB@Lip), where a mass ratio of the lipid film to the hollow mesoporous Prussian blue nanoparticles was 2:1.
(6) 200 mg of an ammonia borane complex (AB) and 100 mg of the liposome-coated hollow mesoporous Prussian blue nanoparticles obtained in step (5) were added to 10 mL of a PBS solution (pH 7.2). A resulting mixture was stirred at 25°C for 24 hours, and loading was conducted. A resulting mixture was centrifuged at 12,000 rpm for 20 minutes, a resulting supernatant was removed, and a resulting precipitate was freeze-dried under vacuum to obtain the Prussian blue composite (HPB@Lip@AB); where a mass ratio of the ammonia borane complex to the liposome-coated hollow mesoporous Prussian blue nanoparticles was 1:0.5.

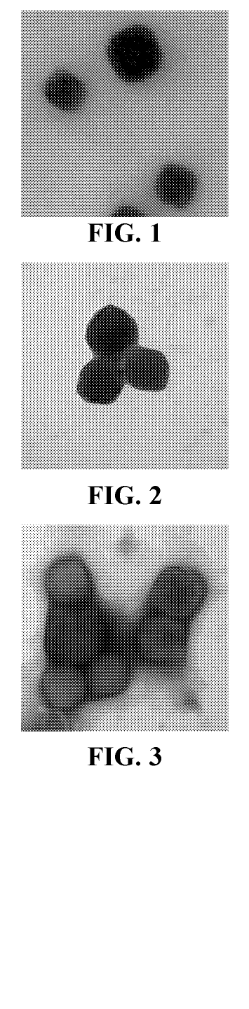

FIG. 1 shows a transmission electron microscope image of the HPB in Example 1; FIG. 2 shows a transmission electron microscope image of the HPB@Lip in Example 1; and FIG. 3 shows a transmission electron microscope image of the HPB@Lip@AB prepared in Example 1.

From FIGS. 1 - 3, it can be seen that the Prussian blue composite consists of hollow mesoporous Prussian blue nanoparticles, a liposome coating the hollow mesoporous Prussian blue nanoparticles, and an ammonia borane complex loaded on the hollow mesoporous Prussian blue nanoparticles.

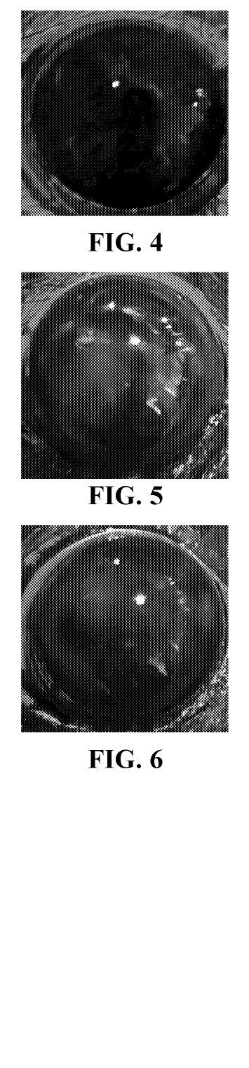

A mouse model of dry eye syndrome was constructed by subcutaneous injection of 0.2 mL of scopolamine hydrobromide (diluting 1.5 mg scopolamine hydrobromide with physiological saline every time, three times per day, for 7 days in total). Then, the HPB@Lip in Example 1, the HPB@Lip@AB prepared in Example 1, and the AB in Example 1 were daily applied to the eyes twice for 14 days, respectively. A slit-lamp was then used for photography, and the photos are shown in FIGS. 4 - 8.

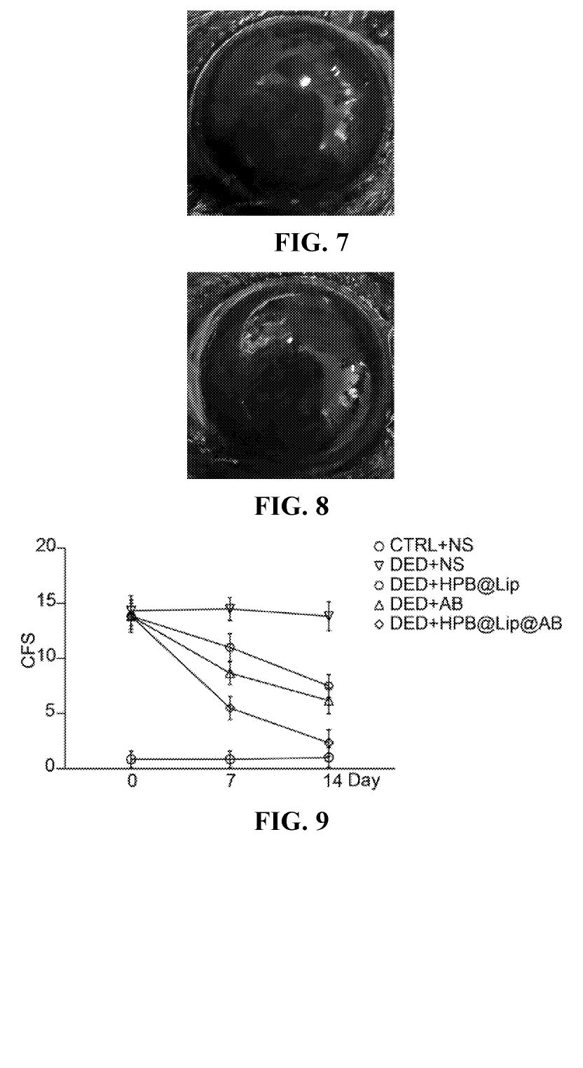

FIG. 4 shows an anterior segment photograph of the corneal sodium fluorescein staining in a normal mouse; FIG. 5 shows an anterior segment photograph of the corneal sodium fluorescein staining in a mouse with dry eye syndrome; FIG. 6 shows an anterior segment photograph of the corneal sodium fluorescein staining in a mouse with dry eye syndrome improved by the HPB@Lip in Example 1; FIG. 7 shows an anterior segment photograph of the corneal sodium fluorescein staining in a mouse with dry eye syndrome improved by the AB in Example 1; and FIG. 8 shows an anterior segment photograph of the corneal sodium fluorescein staining in a mouse with dry eye syndrome improved by the HPB@Lip@AB prepared in Example 1; where, 1 μL of 1% sodium fluorescein was dropwise added to the conjunctival sac of mice, and the corneal staining of each group of mice was observed after 5 minutes.

FIGS. 4 - 8 show that the HPB@Lip@AB prepared in Example 1 significantly improves dry eye symptoms in mice, and has a significantly better effect than that of the HPB@Lip and the AB each.

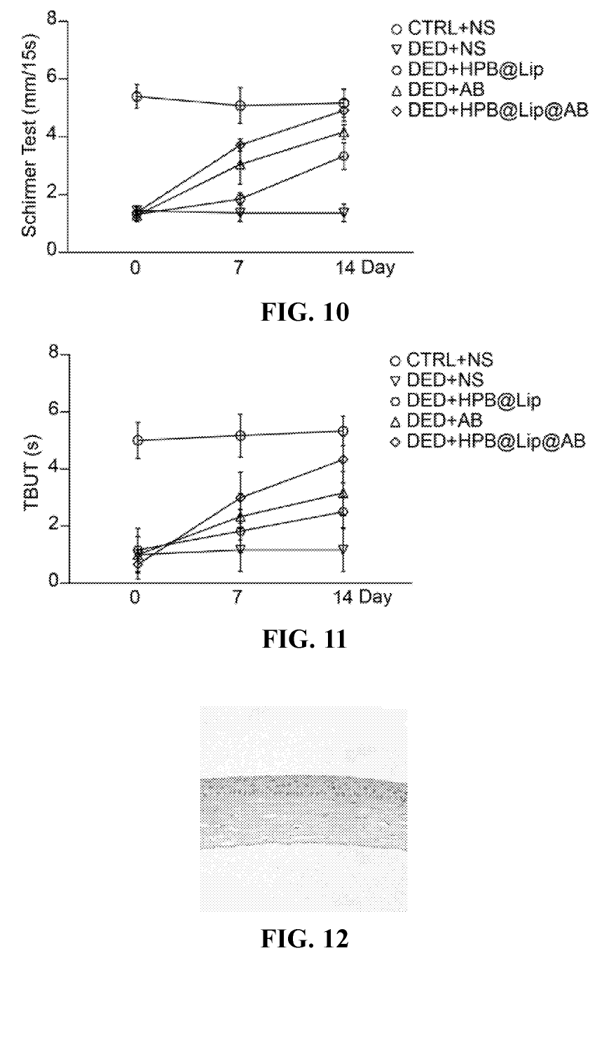

A mouse model of dry eye syndrome was constructed by subcutaneous injection of scopolamine hydrobromide (1.5 mg/0.2 mL/time, three times daily for 7 days in total). Then, 5 μL of normal saline (NS), 5 μL of 200 μg/mL HPB@Lip in Example 1, 5 μL of 200 μg/mL AB in Example 1, and 5 μL of 200 μg/mL HPB@LiP@AB prepared in Example 1 were dropwise added to the eyes. The corneal sodium fluorescein staining scoring of mice is shown in FIG. 9, the secretion amount of tears is shown in FIG. 10, and the tear break-up time is shown in FIG. 11. In the figures, CTRL represents the normal mouse cornea, and DED represents the mouse cornea with dry eye syndrome. 1 μL of 1% sodium fluorescein was dropwise added to the conjunctival sac of mice, and corneal staining was observed in each group of mice after 5 minutes. The grading scale of sodium fluorescein staining employed a 12-point system, that is, the cornea is divided into four quadrants (superior, inferior, nasal, and temporal), with each quadrant scored from 0 to 3 points. No staining is scored 0, 1 to 30 punctate stains are scored 1 point, >30 punctate stains without fusion are scored 2 points, and 3 points indicate the presence of fused punctate stains, filaments, and ulcers in corneas.

From FIGS. 9 - 11, it is seen that compared with the control group, the HPB@Lip@AB has a significant effect in improving dry eye syndrome, which is much better than that of the HPB@Lip and the AB each.

5 μL of 200 μg/mL HPB@Lip in Example 1, 5 μL of 200 μg/mL AB in Example 1, and 5 μL of 200 μg/mL HPB@LiP@AB prepared in Example 1 were applied to the eyes, followed by H&E staining. The results are shown in FIGS. 12 - 16.

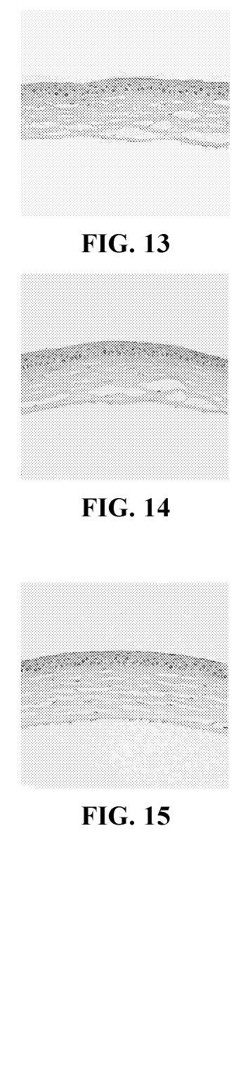

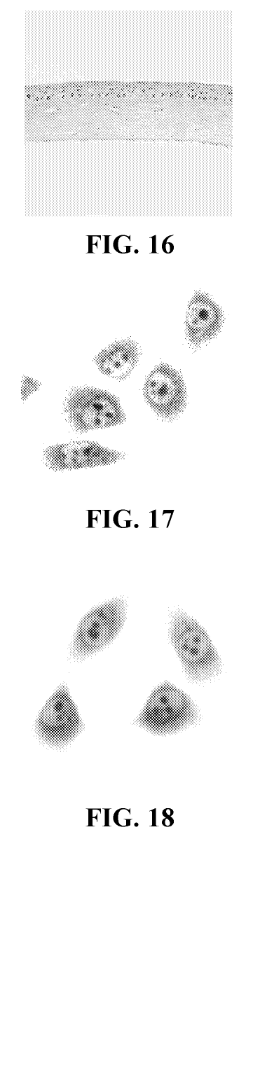

FIG. 12 shows an H&E staining image of the cornea in a normal mouse; FIG. 13 shows an H&E staining image of the cornea in a mouse with dry eye syndrome; FIG. 14 shows an H&E staining image of the mouse cornea applied with the HPB@Lip in Example 1; FIG. 15 shows an H&E staining image of the mouse cornea applied with the AB in Example 1; and FIG. 16 shows an H&E staining image of the mouse cornea applied with the HPB@Lip@AB prepared in Example 1.

The H&E staining images (×400) of mouse corneas show that normal corneas have an intact structure, a dense stromal layer, and a continuous and intact endothelial structure; for corneas with dry eye syndrome, the epithelium is thinner, the stromal layer is disordered, and the endothelial structure is uneven. The application of the HPB@Lip@AB to eyes enables the epithelial thickness, stromal layer, and endothelial structure of corneas to restore, and has a significantly better effect than that of the HPB@Lip and the AB each.

A model of dry eye syndrome was constructed in vitro using a culture system of human corneal epithelial cells (HCECs ) at an osmotic pressure of 550 mOsm·L⁻¹. The cells were incubated with the HPB@Lip in Example 1, the HPB@Lip@AB prepared in Example 1, and the AB in Example 1 at 37°C in a 5% CO₂ incubator for 24 hours, respectively. The results are shown in FIGS. 17 - 22.

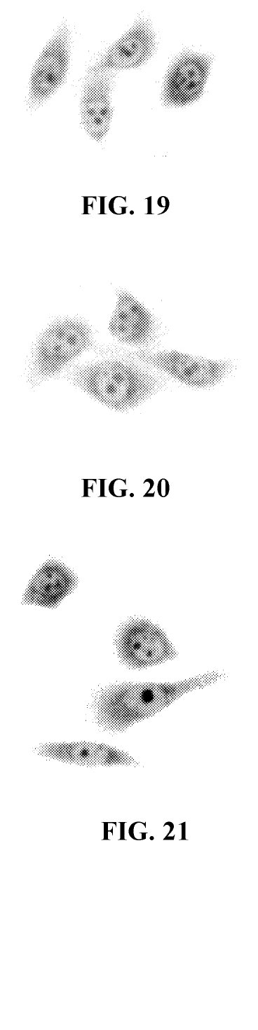

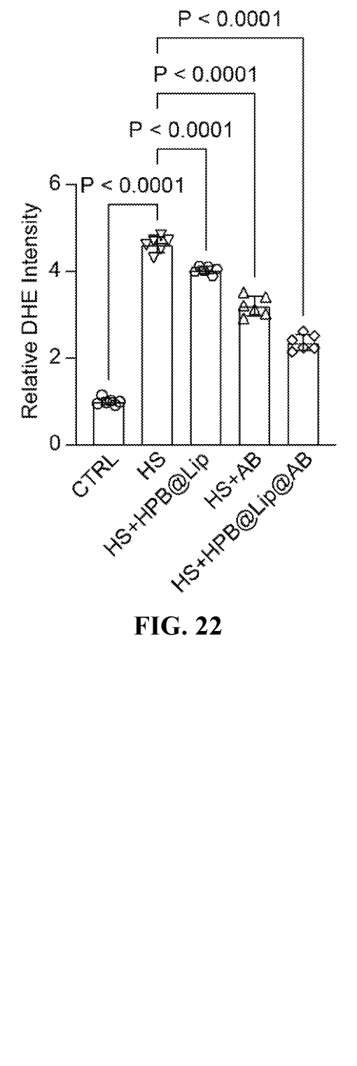

FIG. 17 shows a dihydroethidium (DHE) staining image of normal in vitro HCECs; FIG. 18 shows a DHE staining image of in vitro HCECs with dry eye syndrome; FIG. 19 shows a DHE staining image of in vitro HCECs intervened with the HPB@Lip in Example 1; FIG. 20 shows a DHE staining image of in vitro HCECs intervened with the AB in Example 1; FIG. 21 shows a DHE staining image of in vitro HCECs intervened with the HPB@Lip@AB prepared in Example 1; and FIG. 22 shows the quantitative analysis DHE staining for normal in vitro HCECs, in vitro HCECs with dry eye syndrome, in vitro HCECs intervened with the HPB@Lip in Example 1, in vitro HCECs intervened with the AB in Example 1, and in vitro HCECs intervened with the HPB@Lip@AB prepared in Example 1. In the figures, CTRL represents normal in vitro HCECs, and HS represents in vitro HCECs with dry eye syndrome.

From FIGS. 17 - 22, it is seen that normal in vitro HCECs have a low ROS level; HCECs with dry eye syndrome have a significantly elevated ROS level; the HPB@Lip@AB intervention significantly reduces the ROS level, and has a significantly better effect than that of the HPB@Lip and the AB each. It is indicated that the HPB@Lip@AB can significantly downregulate the ROS level in dry eyes, and has a significantly better effect than that of the HPB@Lip and the AB each.

In summary, in the present disclosure, the Prussian blue composite can effectively improve the symptoms and corneal morphology of mice with dry eye syndrome, and can also reduce the ROS expression level of HCECs in dry eyes, thus effectively improving the symptoms of dry eye syndrome.

The above Example shows that in the present disclosure, the Prussian blue composite can slowly release hydrogen to the inflammatory sites of dry eye syndrome, while alleviating the damage caused by oxidative stress, thereby effectively relieving the symptoms of dry eye syndrome.

The above embodiments are merely preferred embodiments of the present disclosure. It should be noted that a person of ordinary skill in the art may make some improvements and modifications without departing from the principle of the present disclosure, and these improvements and modifications should also be regarded as falling within the scope of the present disclosure.

What is claimed is:

1. A Prussian blue composite comprising hollow mesoporous Prussian blue nanoparticles, a liposome coating the hollow mesoporous Prussian blue nanoparticles, and an ammonia borane complex loaded on the hollow mesoporous Prussian blue nanoparticles.

2. The Prussian blue composite of claim 1, wherein the hollow mesoporous Prussian blue nanoparticles are prepared by a process comprising:
mixing Prussian blue nanoparticles, a first stabilizer, and a first acidic solution to obtain a mixed solution, and subjecting the mixed solution to etching to obtain the hollow mesoporous Prussian blue nanoparticles.

3. The Prussian blue composite of claim 2, wherein the etching is conducted at a temperature of 130°C to 150 °C for 3 hours to 8 hours.

4. A method for preparing the Prussian blue composite of claim 1, comprising steps of:
(1) mixing a liposome raw material and an organic solvent to obtain a lipid solution;
(2) subjecting the lipid solution obtained in step (1) to rotary evaporation to obtain a lipid film;
(3) mixing the lipid film obtained in step (2), the hollow mesoporous Prussian blue nanoparticles, and water, and conducting coating to obtain liposome-coated hollow mesoporous Prussian blue nanoparticles; and
(4) mixing the liposome-coated hollow mesoporous Prussian blue nanoparticles obtained in step (3), a buffer solution, and the ammonia borane complex, and conducting loading to obtain the Prussian blue composite.

5. The method of claim 4, wherein the liposome raw material in step (1) comprises lecithin, cholesterol, and DSPE-mPEG2000 (1,2-distearoyl-sn-glycero-3-phosphoethanolamine-N-[methoxy(polyethylene glycol)-2000]).

6. The method of claim 4, wherein in step (3), a mass ratio of the lipid film to the hollow mesoporous Prussian blue nanoparticles is in a range of 0.5–4 : 1.

7. The method of claim 4, wherein in step (3), the coating is conducted at a temperature of 10°C to 30°C for 10 minutes to 30 minutes.

8. The method of claim 4, wherein in step (4), a mass ratio of the ammonia borane complex to the liposome-coated hollow mesoporous Prussian blue nanoparticles is in a range of 1 : 0.2–17.

9. The method of claim 4, wherein in step (4), the loading is conducted at a temperature of 4°C to 25°C for 24 hours to 72 hours.

10. A method for treating symptoms of dry eye syndrome, comprising administering a therapeutic effective amount of the Prussian blue composite of claim 1 to a subject in need thereof.

11. The method of claim 4, wherein the hollow mesoporous Prussian blue nanoparticles are prepared by a process comprising:
mixing Prussian blue nanoparticles, a first stabilizer, and a first acidic solution to obtain a mixed solution, and subjecting the mixed solution to etching to obtain the hollow mesoporous Prussian blue nanoparticles.

12. The method of claim 11, wherein the etching is conducted at a temperature of 130°C to 150 °C for 3 hours to 8 hours.
